(19) United States
(12) Patent Application Publication
(10) Pub. No.: US 2023/0169240 A1
(43) Pub. Date: Jun. 1, 2023

(54) COMPUTING DEVICE AND METHOD GENERATING OPTIMAL INPUT DATA

(71) Applicant: SAMSUNG ELECTRONICS CO., LTD., Suwon-si (KR)

(72) Inventors: JINWOO KIM, HWASEONG-SI (KR); BYOUNGSEON CHOI, SUWON-SI (KR); YUNJUN NAM, SUWON-SI (KR); SANGHOON MYUNG, GOYANG-SI (KR); JAESIK AN, SEONGNAM-SI (KR); JISU RYU, HWASEONG-SI (KR); CHANGWOOK JEONG, HWASEONG-SI (KR); JAEMYUNG CHOE, SEOUL (KR)

(21) Appl. No.: 17/994,089

(22) Filed: Nov. 25, 2022

(30) Foreign Application Priority Data
Nov. 29, 2021 (KR) 10-2021-0167726

(51) Int. Cl. G06F 30/27

(52) U.S. Cl. CPC G06F 30/27

(57) ABSTRACT

A method of generating optimal input data for a design simulator providing output data related to output parameters in response to input data related to input parameters. The method includes; generating training data including sample input data and sample output data, selecting at least one essential input parameter affecting a plurality of output parameters from among the input parameters in accordance with an estimation model trained using the training data, and generating the optimal input data in accordance with essential input data corresponding to the at least one essential input parameter and the sample output data.

### CROSS-REFERENCE TO RELATED APPLICATION

This application claims priority under 35 U.S.C. §119 to Korean Patent Application No. 10-2021-0167726 filed on Nov. 29, 2021 in the Korean Intellectual Property Office, the subject matter of which is hereby incorporated by reference in its entirety.

### BACKGROUND

The inventive concept relates generally to devices and methods generating optimal input data. More particularly, the inventive concept relates to devices and methods generating optimal input data required to obtain target output data using a design simulator.

Design simulators, such as a technology computer aided design (TCAD) simulator, may be used to estimate one or more characteristics of semiconductor device(s) (hereafter, “semiconductor characteristics”) before, during and/or after fabrication of the semiconductor devices. In order to accurately estimate semiconductor characteristics using a design simulator, observation of output data representing the semiconductor characteristics and calibration of matching output data with target output data is required while varying input data associated with a number of different factors (e.g., semiconductor device layout options, ion implantation options, material(s) attributes and selection, etc.). However, as the number of factors relevant to the definition of semiconductor fabrication process(es) increases with increasing complexity of semiconductor devices, it becomes increasingly difficult and time-consuming to manually perform calibration procedures.

### SUMMARY

Embodiments of the inventive concept provide devices and methods capable of generating optimal input data required to obtain target output data using a design simulator.

According to an aspect of the inventive concept, a computing device may include; a processor, and a memory storing instructions. The processor may be configured to executed the instructions to generate training data that provides output data associated with output parameters in response to input data associated with input parameters, wherein the training data includes sample input data and sample output data, and the processor may be further configured to select an essential input parameter from input parameters in accordance with an estimation model trained using the training data, and generate the optimal input data in relation to essential input data associated with the essential input parameter and the sample output data.

According to an aspect of the inventive concept, a method of generating optimal input data for a design simulator providing output data related to output parameters in response to input data related to input parameters may include; generating training data including sample input data and sample output data, selecting at least one essential input parameter affecting a plurality of output parameters from among the input parameters in accordance with an estimation model trained using the training data, and generating the optimal input data in accordance with essential input data corresponding to the at least one essential input parameter and the sample output data.

According to an aspect of the inventive concept, non-transitory storage medium, when executed by at least one processor, storing instructions for the at least one processor to perform a method generating optimal input data for a design simulator providing output data related to output parameters in response to input data related to input parameters. The method may include; generating training data including sample input data and sample output data, selecting at least one essential input parameter affecting a plurality of output parameters from among a plurality of input parameters in accordance with an estimation model trained using the training data, and generating the optimal input data using essential input data corresponding to the at least one essential input parameter and the sample output data.

### BRIEF DESCRIPTION OF THE DRAWINGS

Advantages, benefits, and/or features, as well as the making and use of the inventive concept, may be more clearly understood upon consideration of the following detailed description together with the accompanying drawings, in which:

FIG. 1 is a flowchart illustrating a method of generating optimal input data according to embodiments of the inventive concept;

FIG. 2 is a conceptual diagram illustrating data flow between a design simulator and an estimation model used in an optimal input data generator according to embodiments of the inventive concept;

FIG. 3 is a flowchart illustrating in one example the method step of generating training data in the method of generating optimal input data of FIG. 1;

FIG. 4 is a table listing possible examples of data generation conditions and corresponding data type that may be used in embodiments of the inventive concept;

FIG. 5 is a flowchart illustrating in one example the method step of selecting at least one essential input parameter in the method of generating optimal input data of FIG. 1;

FIGS. 6 and 7 are conceptual diagrams illustrating an estimation model that may be used in a method of generating optimal input data according to embodiments of the inventive concept;

FIG. 8 is a conceptual diagram illustrating a change amount used in a loss calculation during a method of generating optimal input data according to embodiments of the inventive concept;

FIG. 9 is a flowchart illustrating a method of generating optimal input data based on essential input data in a method of generating optimal input data according to embodiments of the inventive concept;

FIGS. 10 and 11 are respective conceptual diagrams illustrating the sequential and recursive generating of optimal input data in a method of generating optimal input data generation according to embodiments of the inventive concept; and

FIG. 12 is a block diagram illustrating a computing system according to embodiments of the inventive concept.

### DETAILED DESCRIPTION

Throughout the written description and drawings, like reference numbers and labels are used to denote like or similar elements, components, features and/or method steps.

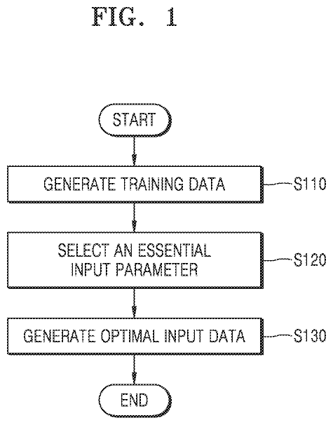

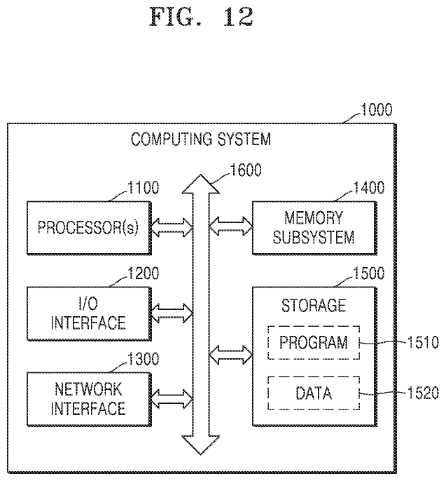

FIG. 1 is a flowchart illustrating a method of generating optimal input data according to embodiments of the inventive concept. In some embodiments, the method of generating optimal data illustrated in FIG. 1 may be performed by an optimal input data generator within a computing system. (See, e.g., the computing system 1000 of FIG. 12).

In this regard, an optimal input data generator may include one or more processor(s) or processing core(s) (hereafter, singularly or collectively, “processor”), as well as non-transitory storage media storing programming code (e.g., code interpreted as various instructions) capable of being executed by the processor. That is, the processor may be configured to execute (or perform) various instructions that generate optimal input data for a design simulator, wherein the design simulator provides output data (e.g., data including or related to output parameters) in response to input data (e.g., data including or related to input parameters).

Further in this regard, the optimal input data generator may be implemented as a combination of hardware, firmware and/or software resources (e.g., one or more modules), such that the processor is capable of executing one or more operations (or method steps) that collectively generate optimal input data. When implemented, wholly or in part, as a software module, related functions may be stored as instruction(s) in a tangible non-transitory storage medium. Exemplary software module(s) may be included in random access memory (RAM), flash memory, read-only memory (ROM), electrically programmable ROM (EPROM), electrically erasable and programmable ROM (EEPROM), register(s), a hard disk, a mobile disk, compact disk (CD) ROM, etc.

Optimal input data may be understood as data that is input (or applied) to the design simulator in order to obtain target output data (e.g., data resulting in the target data in response to operation of the design simulator).

In some embodiments, the design simulator may include one or more physical model(s) designed to emulate an actual environment. That is, the design simulator performs a simulation on input data in order to provide output data (e.g., simulation results). As will be appreciated by those skilled in the art, the design simulator may operate in response to one or more mathematical formulas, computational theories and/or physical principles. For example, in some embodiments, a technology computer aided design (TCAD) simulator or an electronic computer aided design (ECAD) simulator may be used.

Consistent with the foregoing, in some embodiments, a design simulator may perform simulation(s) in response to input data, wherein the input data relates to one or more variables (e.g., layout variables, design variable, processing variables, etc.). Accordingly, the input data may include a number of input parameters corresponding to the variables.

Output data (e.g., a simulation result) generated by (or resulting from operation of) the design simulator may include data relating to, for example, the electrical, mechanical, structural and/or materials characteristics of a semiconductor device (hereafter, singularly of collectively, “semiconductor characteristics”). Accordingly, the output data may include a number of output parameters corresponding to the semiconductor characteristics.

In order to obtain the semiconductor characteristics, a semiconductor device designer may define (or set) optimal input data required to obtain target output data associated with semiconductor characteristics using the design simulator. In this regard, the designer may adjust (e.g., manipulate) the input data applied to the design simulator and perform various calibration processes that match resulting output data with the target output data. Further in this regard, when the designer manually adjusts the input data during calibration, the overall calibration process may take a long time. This is particularly true when a large number of factors are simultaneously considered. Accordingly, accuracy (or consistency) of the calibration outcome may be reduced.

Referring to the method of generating optimal input data illustrated in FIG. 1, training data may be generated (S110). Here, the term “training data” denotes data used to train an estimation model, as described hereafter, and may include sample input data and/or sample output data, wherein sample input data is one example of the input data, and sample output data is one example of the output data corresponding to the sample input data. Thus, when sample input data is applied to the design simulator, the design simulator may output the sample output data in response to the sample input data.

Here, the term “estimation model” denotes one or more model(s) generated by emulating the operation of a design simulator. When a simulation is performed using the estimation model, a simulation result may be obtained in less time than when the same simulation is performed using the design simulator. However, the estimation model must be properly trained (e.g., using training data) to improve its consistency.

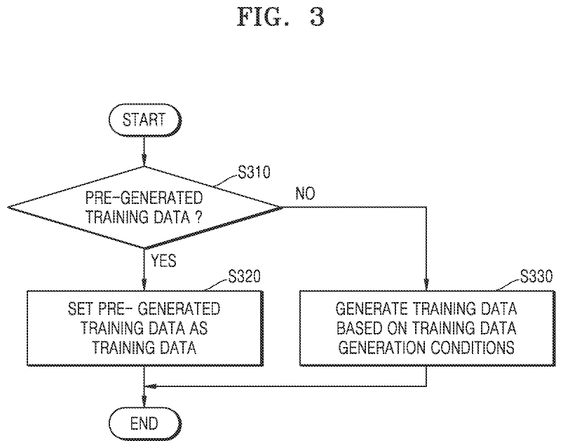

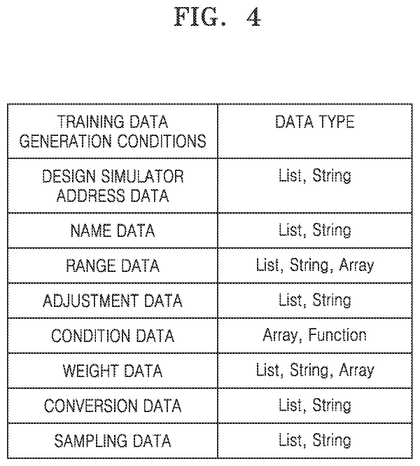

In this regard, training data may be generated by use of the design simulator in relation to training data generation conditions, wherein training data generation conditions may include information associated with sample input data and/or sample output data, constraints on sample input data, constraints of sample output data, etc. An exemplary method of generating training data will be described hereafter in some additional detail in relation to FIG. 3, and one example of training data generation conditions is listed in FIG. 4.

Once the training data has been generated, an essential input parameter (e.g., at least one essential input parameter) may be selected (S120). Here, in some embodiments, an essential input parameter may be understood as an input parameter selected from a number of input parameters that affects two or more output parameters.

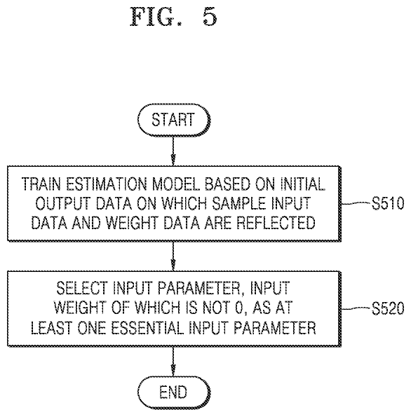

The essential input parameter may be selected from among other input parameters using the estimation model, once trained through machine learning using the training data. In some embodiments, the essential input parameter may be selected in relation to an input weight indicating a number of output parameters affected by an input parameter. An exemplary method of selecting the essential input parameter using an estimation model will be described hereafter in some additional detail in relation to FIGS. 5, 6, 7, and 8.

Once the essential input parameter has been selected, optimal input data may be generated (S130). Here, the term “optimal input data” may be understood as data that must be applied (e.g., required input data) to the design simulator in order to obtain the target output data using the design simulator.

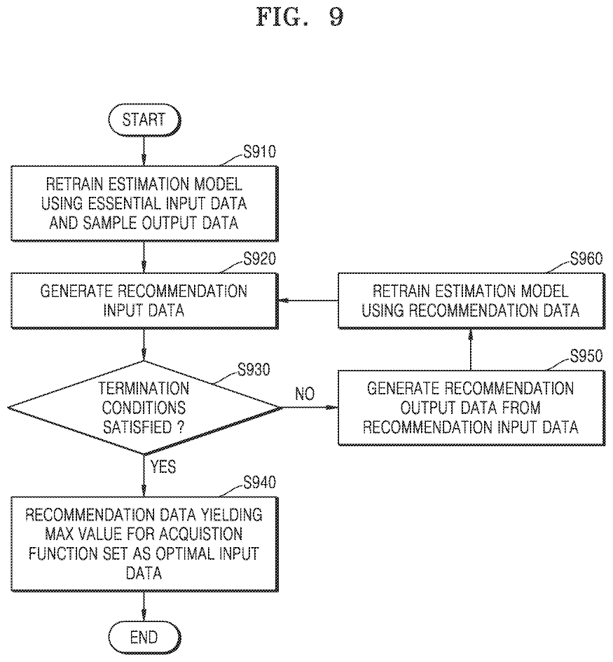

In some embodiments, the estimation model may be retrained in relation to essential input data and sample output data. Alternately or additionally, recommendation input data may be generated in relation to values of an acquisition function according to a retrained estimation model. In some embodiments, the design simulator may be run (e.g., the design simulation may be performed) in accordance with a determination of whether certain termination conditions have been satisfied. In some embodiments, optimal input data may be generated in accordance with recommendation input data. An exemplary method of generating optimal input data will be described hereafter in some additional detail in relation to FIG. 9.

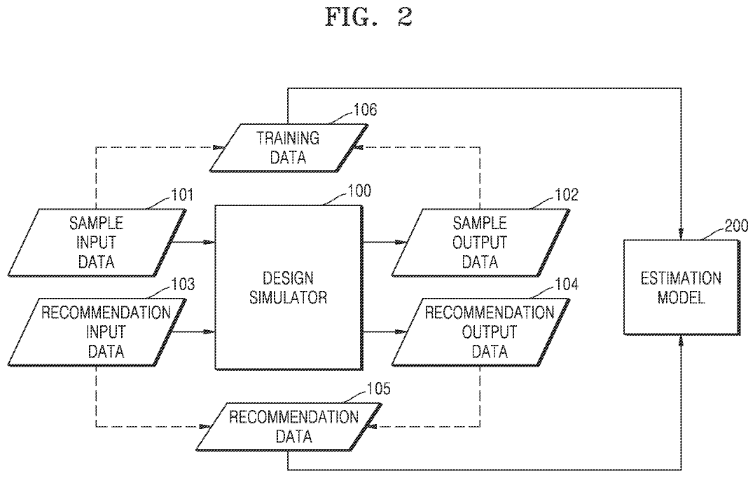

FIG. 2 is a conceptual diagram illustrating an exemplary flow of data between a design simulator 100 and an estimation model 200 during operation of a optimal input data generator consistent with embodiments of the inventive concept.

Referring to FIG. 2, the design simulator 100 may output sample output data 102 in response to sample input data 101. That is, sample output data 102 may be generated by the design simulator 100 based on (or in accordance with) sample input data 101. Thus, in some embodiments, sample input data 101 and sample output data 102 may be included in training data 106.

Alternately or additionally, the design simulator 100 may output recommendation output data 104 in response to recommendation input data 103. That is, in some embodiments, recommendation output data 104 may be generated by the design simulator 100 based on the recommendation input data 103. Thus, in some embodiments, recommendation input data 103 and recommendation output data 104 may be included in recommendation data 105.

Accordingly, training data 106 and recommendation data 105—as generated by the design simulator 100—may be input to the estimation model 200, and the estimation model 200 may be trained in accordance with the training data 106 and the recommendation data 105.

FIG. 3 is a flowchart further illustrating in one example the step of generating training data (S110) in the method of generating optimal input data of FIG. 1.

Referring to FIG. 3, a threshold determination may be made as to whether pre-generated training data exists (S310). In this regard, when there is a history of generating optimal input data using existing training data, pre-generated training data may exist.

If pre-generated training data exists (S310=YES), no training data need be generated, and the pre-generated training data may be set as training data (S320). However, if pre-generated training data does not exist (S310=NO), training data will be generated in accordance with training data generation conditions (S330). For example, training data may be generated by performing a design simulation in relation to the training data generation conditions.

FIG. 4 is a table listing by way of example possible training data generation conditions, and corresponding data type, that may be used during generation of optimal input data according to embodiments of the inventive concept.

Referring to FIG. 4, the training data generation conditions may include, at least one of for example; design simulator address data, name data, range data, adjustment data, condition data, weight data, conversion data, and/or sampling data.

The design simulator address data may include information indicating an address used to bring the design simulator executed at the time of generating the training data. The design simulator address data may have a data type of List or String.

The name data may include information indicating names of input parameters and output parameters which may be included in the input data and the output data, respectively. The name data may have a data type of List or String.

The range data may include information indicating a range of values for the input parameters, and may correspond to name data for the input parameters. For example, range data may include information indicating that a first input parameter is required to be about 0.1 to about 1. Here, the range data may have a data type of List, String, or Array.

The adjustment data may include information indicating a reference for adjusting values of the input parameters at the time of executing the design simulator for generating the training data, and may correspond to name data for the input parameters. For example, the adjustment data may include information indicating that the first input parameter may be adjusted by about 0.1. Here, the adjustment data may have a data type of List or String.

The condition data may include information indicating conditions to be satisfied between the input parameters. For example, the condition data may include information indicating that the first input parameter is required to have a greater value than a second input parameter. The condition data may have a data type of Array or Function.

The weight data may include information indicating relative priority of the output parameters. For example, the weight data may include information indicating that a first output parameter has a higher priority than a second output parameter. The weight data may have a data type of List, String, or Array.

The conversion data may include information indicating a conversion method for reducing a learning error according to size differences between values of the input parameters and/or the output parameters. The conversion data may have a data type of List or String.

The sampling data may include information indicating which sampling technique (or approach) should be used to perform sampling at the time of generating the training data. For example, the sampling data may include information indicating random sampling, Latin hypercube sampling, constrained sampling, etc. Here, the sampling data may have a data type of List or String.

Referring to FIG. 3, training data may be generated (S330) by performing a design simulation (e.g., executing the design simulator) in accordance with training data generation conditions, as described in FIG. 4. In some embodiments, sample input data may be generated in accordance with name data, range data, adjustment data, condition data, and/or sampling data, and the training data may be generated by generating sample output data corresponding to the sample input data using the design simulator based on the design simulator address data.

FIG. 5 is a flowchart further illustrating in one example the step of selecting at least one essential input parameter (S120) in the method of generating optimal input data of FIG. 1.

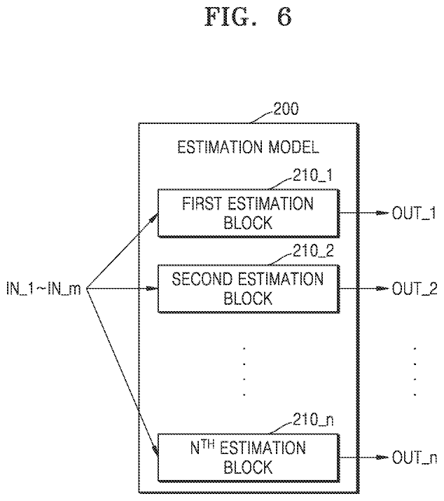

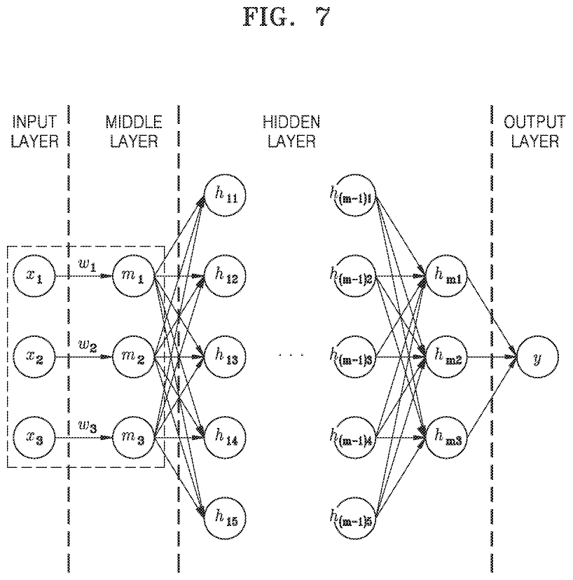

Referring to FIG. 5, the estimation model may be trained based on initial output data on which sample input data and the weight data are reflected (S510). Then, one or more input parameter(s) may be selected as corresponding essential input parameter(s) given a non-zero input weight (S520). One example of the estimation model trained according to this approach will be described hereafter in some additional detail in relation to FIGS. 6 and 7.

FIG. 6 is a block diagram further illustrating in one example the estimation model 200 of FIG. 2.

Referring to FIG. 6, the estimation model 200 may include first through n^{th} estimation blocks 210_1 through 210_n.

Each of the first through n^{th} estimation blocks 210_1 through 210_n may output one or more of first through n^{th} output parameters OUT_1 through Out_n in response to one or more of input parameters IN_1 through IN_m. That is, each of the first through n^{th} estimation blocks 210_1 through 210_n may output one or more of first through n^{th} output parameters OUT_1 through OUT_n by performing a calculation based on one or more of the input parameters IN_1 through IN_m.

For example, the first estimation block 210_1 may output the first output parameter OUT_1 in response to input parameters IN_1 through IN_m. The second estimation block 210_2 may output the second output parameter OUT_2 in response to the input parameters IN_1 through IN_m. The nth estimation block 210_n may output the n^{th} output parameter OUT_n in response to the input parameters IN_1 through IN_m.

In some embodiments, each of the first through n^{th} estimation blocks 210_1 through 210_n may have a neural network structure. For example, each of the first through n^{th} estimation blocks 210_1 through 210_n may have a structure corresponding to any one of a deep neural network (DNN), a convolution neural network (CNN), a recurrent neural network (RNN), etc. One example of an estimation block that may be used in the embodiment of FIG. 6 is illustrated in some additional detail in FIG. 7.

FIG. 7 is a conceptual diagram illustrating an estimation block that may be used in a method of generating optimal input data according to embodiments of the inventive concept.

Referring to FIG. 7, the estimation block may include an input layer, a middle layer, a hidden layer, and an output layer.

The input layer may receive the input data including input parameters. That is, the input layer may include the number of nodes corresponding to the number of input parameters.

For example, assuming a total number of input parameters is three, the input layer may include a first input node x₁, a second input node x₂, and a third input node x₃. In this case, the first input parameter may be input to the first input node x₁, the second input parameter may be input to the second input node x₂, and a third input parameter may be input to the third input node x₃.

The input layer may apply first, second and third input weights w₁, w₂, and w₃ to the received data, and output the corresponding results to the middle layer. That is, the input layer may respectively apply (e.g., multiply) the first, second and third weights w₁, w₂, and w₃ to each of the received input parameters to generate and output corresponding results to the middle layer. In the illustrated example of FIG. 7, the first input node x₁ may multiply the first input weight w₁ by the first input parameter and output the result thereof, the second input node x₂ may multiply the second input weight w₂ by the second input parameter and output the result thereof, and the third input node x₃ may multiply the third input weight w₃ by the third input parameter and output the result thereof.

The middle layer may receive the values obtained by multiplying the first, second and third input weights w₁, w₂, and w₃ by the input parameters from the input layer. In this case, the middle layer may include the same number of nodes as the input layer, and nodes included in the middle layer may be connected to nodes included in the input layer one-to-one.

In the illustrated example of FIG. 7, the middle layer may include a first middle node m₁, a second middle node m₂, and a third middle node m₃. In this case, the first middle node m₁ may be connected to the first input node x₁, the second middle node m₂ may be connected to the second input node x₂, and the third middle node m₃ may be connected to the third input node x₃.

The middle layer may apply various weights to values received from the input layer, and output the result thereof to the hidden layer.

The hidden layer may include a number of layers, and may be connected to the middle layer. In the illustrated example of FIG. 7, the hidden layer may include ‘k’ layers, and among the k layers, nodes h₁₁, h₁₂, h₁₃, h₁₄, and h₁₅ included in a first hidden layer may be connected to the first, second and third nodes m1, m2, and m₃ included in the middle layer. Each of the nodes h₁₁, h₁₂, h₁₃, h₁₄, and h₁₅ included in the first hidden layer may be connected to each of the first through third nodes m₁, m2 and m₃ included in the middle layer. In this case, each of the first, second and third nodes m₁, m2 and m₃ included in the middle layer may apply different weights from each other to values received from the input layer, and output the result thereof to each of the nodes h₁₁, h₁₂, h₁₃, h₁₄, and h₁₅.

Each of the nodes h₁₁, h₁₂, h₁₃, h₁₄, and h₁₅ included in the first hidden layer may apply different weights from each other to a value obtained by summing values received from the middle layer, and output the result thereof to nodes included in a second hidden layer. In addition, each of the nodes included in the second hidden layer may apply different weights to a value obtained by summing values received from the first hidden layer, and output the result thereof to nodes included in a third hidden layer. By using such a process, finally, nodes included in a k^{th} hidden layer may receive values from nodes included in a (k-1)^{th} hidden layer.

The output layer may be connected to the hidden layer. In the illustrated example of FIG. 7, the output layer may be connected to nodes hₘ₁, hₘ₂, and hₘ₃ included in an m^{th} hidden layer. In this case, the output layer may include a single node. The output layer may receive values from the nodes hₘ₁, hₘ₂, and hₘ₃ included in the m^{th} hidden layer, and by summing all of received values, may calculate an output value for the estimation block. In addition, the output value of the estimation model may include any one value among the output parameters.

Referring to FIG. 5, the estimation model of the type described above in relation to FIGS. 6 and 7 may be trained (S510), and each of a number of estimation blocks may be trained to output any one of the output parameters on which weight data has been reflected in response to the input parameters included in the sample input data.

The estimation model 200 may be trained in response to sample input data so that a loss between sample estimation output data generated by the estimation model and the sample output data may be reduced.

This loss may be calculated as a difference between the sample estimation output data and the sample output data, and a difference between a change of the output parameters included in the sample estimation output data and a change of the output parameters included in the sample output data.

The difference between the sample estimation output data and the sample output data may be calculated as a difference between the output parameters included in the sample estimation output data and the output parameters included in the sample output data. For example, the difference between the sample estimation output data and the sample output data may include a difference between the first output parameter of the sample estimation output data and the first output parameter of the sample output data through a difference between an m^{th} output parameter of the sample estimation output data and the m^{th} output parameter of the sample output data.

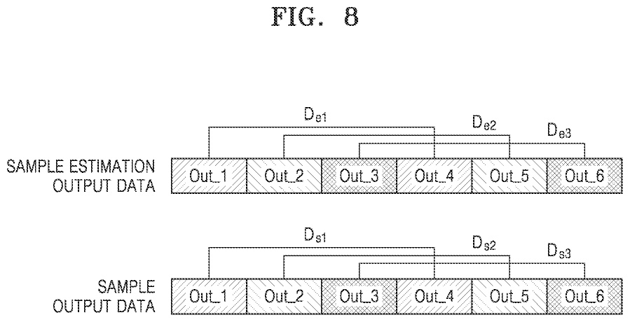

One approach to determining the difference between a change of the output parameters included in the sample estimation output data and a change of the output parameters included in the sample output data may be described hereafter in some additional detail in relation to FIG. 8.

FIG. 8 is a conceptual diagram illustrating a “change amount” used in a loss calculation related to a method of generating optimal input data according to embodiments of the inventive concept.

Referring to FIG. 8, six (6) examples of the sample estimation output data and the sample output data are assumed. That is, the sample estimation output data and the sample output data each include first, second, third, fourth, fifth and sixth output parameters Out_1 through Out_6.

Under these assumptions, change amounts for output parameters included in the sample estimation output data may include a first estimation change amount Dₑ₁, which is a difference between the first output parameter Out_1 and the fourth output parameter Out_4, a second estimation change amount Dₑ₂, which is a difference between the second output parameter Out_2 and the fifth output parameter Out_5, and a third estimation change amount Dₑ₃, which is a difference between the third output parameter Out_3 and the sixth output parameter Out_6.

In addition, change amounts of the output parameters included in the sample output data may include a first sample change amount Dₛ₁, which is a difference between the first output parameter Out _1 and the fourth output parameter Out_4, a second sample change amount Dₛ₂, which is a difference between the second output parameter Out_2 and the fifth output parameter Out_5, and a third sample change amount Dₛ₃, which is a difference between the third output parameter Out_3 and the sixth output parameter Out_6.

A difference between change amounts of the output parameters included in the sample estimation output data and change amounts of the output parameters included in the sample output data may include a difference between the first estimation change amount Dₑ₁ and a first sample change amount Dₛ₁, a difference between the second estimation change amount Dₑ₂ and the second sample change amount Dₛ₂, and a difference between the third estimation change amount Dₑ₃ and the third sample change amount Dₛ₃.

Those skilled in the art will recognize that the illustrated example of FIG. 8 is just that; an example, and the scope of the inventive concept is not limited thereto.

Referring to FIG. 5, loss may be calculated based on the calculated differences described above. Further in this regard, loss may be calculated using an approach, such as for example, mean absolute error (MAE), mean squared error (MSE), root mean squared error (RMSE), etc.

The estimation model may be trained such that loss, of the type described above, may be reduced. That is, the estimation model may generate the sample estimation output data in response to the sample input data, may be trained such that loss calculated using the sample estimation output data and the sample output data is reduced, while input weights and weights between nodes included in a number of estimation blocks.

After the estimation model has been trained, the input parameter having a non-zero input weight may be selected as an essential input parameter. That is, when the input weight applied at the time when the input parameter is output from the input layer to the middle layer of the estimation block is not about zero, the input parameter may be selected as the essential input parameter.

FIG. 9 is a flowchart further illustrating in one example the step of generating optimal input data (S130) in the method of generating optimal input data of FIG. 1.

Referring to FIG. 9, an estimation model may be retrained in accordance with the essential input data and the sample output data (S910).

In some embodiments, retraining of the estimation model (S910) may be substantially similar to the estimation model training of (S510), with the exception that the estimation model retraining of method step (910) is performed in relation to essential input data, and not sample input data. That is, during the method step of (S910), the estimation model will not be retrained in relation to input parameters not selected as essential input parameters.

After the estimation model has been retrained, recommendation input data may be generated (S920).

The recommendation input data may respectively be candidates for the optimal input data required for obtaining target output data using the retrained estimation model. In some embodiments, recommendation input data may be generated based on values of an acquisition function according to the retrained estimation model. Here, the acquisition function may include a function stochastically indicating which type of input data is required to be input to the estimation model to output most similar output data as target output data, and may use any one of a probability of improvement (PI) function, an expected improvement (EI) function, etc.

Because the optimal input data associated with the target output data must ultimately be generated, pre-set input data which maximizes the values of the acquisition function according to the estimation model may be generated as the recommendation input data.

After the recommendation input data has been generated, a determination may be made as to whether certain termination conditions have been satisfied (S930).

Here, termination conditions may include conditions as a reference for determining whether the optimal input data may be generated without further retraining of the estimation model. The termination conditions may include at least one conditions such as whether the number of retraining operations exceeds a preset reference number, whether the loss calculated during the retraining process of the estimation model is less than the preset reference value, etc. In response to the determination of whether the termination conditions have been satisfied, the design simulation may be run or the optimal input data may be generated based on the recommendation input data.

That is, if the termination conditions have been satisfied (S930=YES), the recommendation input data, in which the value of the acquisition function has the maximum value, from among the recommendation input data may be generated as the optimal input data (S940). Here, because the acquisition function stochastically indicates whether the output data most similar to the target output data is to be output, the recommendation input data, in which the value of the acquisition function has the maximum value from among the recommendation input data may include the input data most appropriate for obtaining the target output data. Accordingly, the recommendation input data yielding maximum value for the acquisition function from among the recommendation input data may be selected (or set) as the optimal input data.

Alternately, if the termination conditions have not been satisfied (S930=NO), the design simulation may be run using recommendation input data in order to generate recommendation output data (S950).

Here, the recommendation output data may include data output by the design simulator in response to the recommendation input data. Accordingly, when the recommendation input data are input to the design simulator, the design simulator may output the recommendation output data respectively corresponding to the recommendation input data. Accordingly, the recommendation data may be generated which includes the recommendation input data and the recommendation output data respectively corresponding to the of recommendation input data.

Once the recommendation output data are generated, the estimation model may be retrained in accordance with the recommendation data (S960).

In some embodiments, the method step of retraining the estimation model (S960) may be substantially similar to the method step of training the estimation model (S910). However, the retraining of the estimation model may be performed in relation to recommendation input data and recommendation output data, instead of essential input data and the sample output data. That is, the estimation model may be retrained using data that has been newly generated by the design simulator.

After the estimation model has been retrained using recommendation data, method steps (S920) and (S930) may again be performed. That is, after the estimation model has been retrained using recommendation data, the recommendation input data may be generated based on the values of the acquisition function according to the retrained estimation model (S920). In addition, the determination of whether the termination conditions have been satisfied may again be made (S930), and in accordance with this determination, the design simulation may be run using the design simulator, or the optimal input data may be generated based on the recommendation input data.

The process of generating the optimal input data as described in relation to FIG. 9 may be performed sequentially or recursively for each of a number of input parameter groups including at least one essential input parameter.

An input parameter group may include, for example, input parameters having identical physical characteristics from among the input parameters. For example, in some embodiments, a first input parameter group may include the input parameters related to a mask used during the fabrication of a semiconductor device, and a second input parameter group may include input parameters related to a mold used during the fabrication of the semiconductor device.

Each of the input parameters may be classified according to one of the input parameter groups, and each of the output parameters may be classified according to one of output parameter groups. Here, in some embodiments, the output parameter groups may respectively correspond to the input parameter groups. In addition, each estimation block may be classified according to one of a number of estimation block groups, and the estimation block groups may respectively correspond to the output parameter groups.

Accordingly, a method of generating optimal input data may be sequentially or recursively performed according to whether correlations exist between the input parameter groups. In this regard, a correlation may be expressed an index indicating whether physical characteristics indicated by each of the input parameter groups affect one another. Alternately or additionally, a designed may preset one or more correlations between the input parameter groups.

In some embodiments wherein there is no correlation between the input parameter groups, the method of generating optimal input data may be sequentially performed. Alternately, when one or more correlations exist between the input parameter groups, the method of generating optimal input data may be recursively performed.

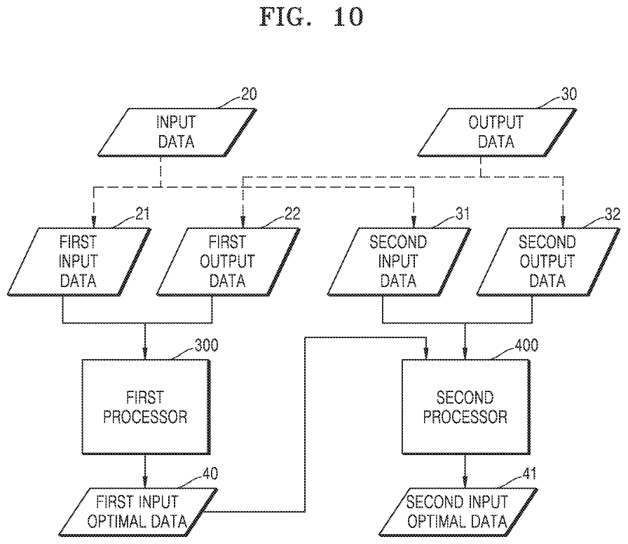

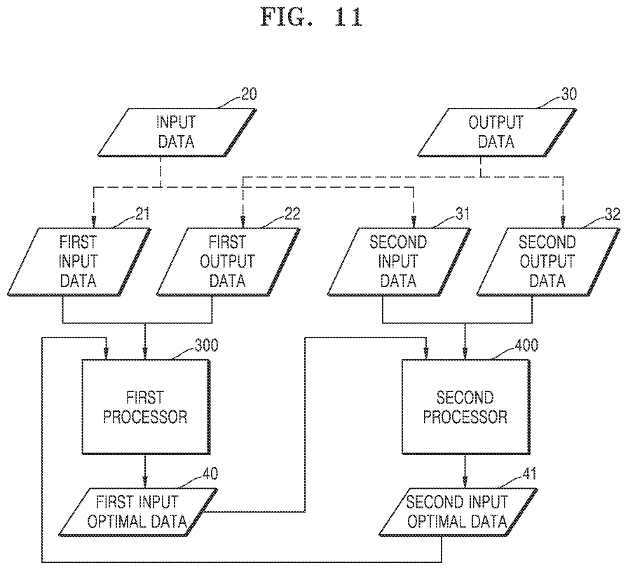

Methods of generating optimal input data, either sequentially or recursively, for each input parameter group will be comparatively described in relation to FIGS. 10 and 11.

FIG. 10 is a block diagram illustrating a method of sequentially generating optimal input data according to embodiments of the inventive concept.

Referring to FIG. 10, input data 20 including input parameters may be classified into first input data 21 including one or more input parameters included in the first input parameter group, and second input data 31 including one or more input parameters included in the second input parameter group. In addition, the output data 30 may include first output data 22 corresponding to one or more output parameters included in the first output parameter group, second output data 32 corresponding to one or more output parameters included in the second output parameter group.

Here, the first input data 21 and the first output data 22 may be input to a first processor 300. Then, the first processor 300 may retrain a first estimation block group corresponding to the first input parameter group and the first output parameter group based on the first input data 21 and the first output data 22, and generate the first optimal input data 40 corresponding to the optimal input data based on the retrained first estimation block group.

Next, the first optimal input data 40 output by the first processor 300, the second input data 31, and second output data 32 may be input to a second processor 400. Then, the second processor 400 may retrain a second estimation block corresponding to the second input parameter group and the second output parameter group based on the first optimal input data 40 output by the first processor 300, the second input data 22, and the second output data 31, and may generate the sample input data corresponding to second optimal input data 41 based on the second estimation block group.

In this manner, generation of the optimal input data for the first input parameter group, and generation of the optimal input data for the second input parameter group may sequentially proceed.

FIG. 11 is a block illustrating a method of recursively generating optimal input data according to embodiments of the inventive concept.

Referring to FIG. 11, the method of recursively generating the optimal input data is substantially similar to the method of sequentially generating optimal input data of FIG. 10, except after the second optimal input data 41 is generated, the second optimal input data 41, the first input data 21, and the first output data 22 may be applied to the first processor 300. Then, the first processor 300 may retrain the first estimation block group based on the first input data 21, the -first output data 22, and the second optimal input data 41 in order to generate the first optimal input data 40.

That is, because there are correlations between the first input parameter group and the second input parameter group, after the sample input data corresponding to the optimal input data is generated, retraining on the first estimation block group may be performed. Accordingly, the first input data corresponding to the optimal input data may be regenerated. Similarly, after the first input data corresponding to the optimal input data is regenerated, retraining on the second estimation block group may be performed, and the second input data corresponding to the optimal input data may be regenerated. By recursively performing these method steps, the optimal input data may generated.

In some embodiments, the first processor 300 and the second processor 400 are separately provided in relation to FIGS. 10 and 11. Alternately, the first processor 300 and the second processor 400 are implemented using a single processor.

FIG. 12 is a block diagram illustrating a computing system 1000 according to embodiments of the inventive concept. In some embodiments, a method of generating optimal input data, like the ones previously described, may be performed using the computing system 1000 of FIG. 12. Here, the computing system 1000 may be implemented as a stationary computing system such as a desktop computer, a workstation, and a server, or as a portable computing system such as a laptop computer.

Referring to FIG. 12, the computing system 1000 may include at least one processor 1100, an input/output (I/O) interface 1200, a network interface 1300, a memory subsystem 1400, and a storage 1500, and the at least one processor 1100, the I/O interface 1200, the network interface 1300, the memory subsystem 1400, and the storage 1500 may communicate with each other via a bus 1600.

The at least one processor 1100 may be referred to as a processing unit, and like a micro-processor, an application processor (AP), a digital signal processor (DSP), and a graphics processing unit (GPU), may execute an arbitrary set of instructions (e.g., Intel Architecture-32 (IA-32), 64-bit extension IA-32, x86-64, PowerPC, scalable processor architecture (SPARC), microprocessor without interlocked pipeline stages (MIPS), and Acorn reduced instruction set computer (RISC) machine (ARM), Intel Architecture-62 (IA-64), etc.). For example, the at least one processor 1100 may access the memory subsystem 1400 via the bus 1600, and execute instructions stored in the memory subsystem 1400. In some embodiments, when there are a number of processors 1100, method steps of selecting at least one essential input parameter may be performed in parallel for a number of estimation blocks using the processors 1100.

The I/O interface 1200 may include an input device such as a keyboard and a pointing device, and/or an output device such as a display device and a printer, or may provide access to the input device and/or the output device. A user may trigger execution of a program 1510 and/or loading of data 1520, may also input the training data generation conditions in FIG. 4, and may also identify the generated optimal input data.

The network interface 1300 may provide access to a network outside the computing system 1000. For example, the network may include a plurality of computing systems and communication links, and the communication links may include wired links, optical links, wireless links, or any other types of links.

The memory subsystem 1400 may store the program 1510 for a method of modeling a loss described above with reference to the drawings or at least a portion thereof, and the at least one processor 1100 may perform at least a portion of operations included in the method of generating the optimal input data by executing programs (or instructions) stored in the memory subsystem 1400. The memory subsystem 1400 may include ROM, RAM, etc.

The storage 1500 may include a non-transitory storage medium, and may not lose data stored therein even when power supplied to the computing system 1000 is cut off. For example, the storage 1500 may also include a non-volatile memory device, and may also include a storage medium such as magnetic tape, an optical disk, and a magnetic disk. In addition, the storage 1500 may also be detachable from the computing system 1000. As illustrated in FIG. 12, the program 1510 and the data 1520 may be stored. Before being executed by the at least one processor 1100, at least a portion of the program 1510 may be loaded in the memory subsystem 1400. In some embodiments, the storage 1500 may store a file coded in a program language, and the program 1510 generated from the file by a compiler or at least a portion thereof may be loaded in the memory subsystem 1400. The data 1520 may include the training data, the recommendation data, the optimal input data, etc.

According to a method of generating optimal input data and related system(s) described above, optimal input data may be automatically (i.e., non-manually) generated in relation to at least one essential input parameter based on the estimation model trained by the machine learning. In addition, calibration may be performed with greater consistency while consuming less time, as compared with manually performed calibration techniques.

While the inventive concept has been particularly shown and described with reference to embodiments thereof, it will be understood that various changes in form and details may be made therein without departing from the spirit and scope of the following claims.

What is claimed is:

1. A computing device comprising:
a processor; and
a memory storing instructions,
wherein the processor is configured to executed the instructions to generate training data that provides output data associated with output parameters in response to input data associated with input parameters,
the training data includes sample input data and sample output data, and
the processor is further configured to select an essential input parameter from input parameters in accordance with an estimation model trained using the training data, and generate the optimal input data in relation to essential input data associated with the essential input parameter and the sample output data.

2. The computing device of claim 1, wherein the processor is further configured to determine whether pre-generated training data exists, and
if the pre-generated training data exists, set the pre-generated training data as the training data, else generate the training data in relation to training data generation conditions.

3. The computing device of claim 2, wherein the processor is further configured to generate the training data by executing a design simulation using the design simulator in relation to the training data generation conditions.

4. The computing device of claim 2, wherein the training data generation conditions include at least one of design simulator address data, name data, range data, adjustment data, condition data, weight data, conversion data, and sampling data.

5. The computing device of claim 4, wherein the essential input parameter has a corresponding non-zero input weight.

6. The computing device of claim 1, wherein the processor is further configured to train the estimation model using the sample input data and the sample output data, on which weight data is reflected, and select the essential input parameter.

7. The computing device of claim 6, wherein the processor is further configured to train the estimation model based on a loss between a sample estimation output data generated by the estimation model in response to the sample input data and the sample output data.

8. The computing device of claim 7, wherein the processor is further configured to calculate the loss based on a difference between the sample estimation output data and the sample output data, and a difference between a change amount of the output parameters associated with the sample estimation output data and a change amount of the output parameters associated with the sample output data.

9. The computing device of claim 6, wherein the estimation model includes estimation blocks, wherein each estimation block among the estimation blocks provides an output parameter from among the output parameters in response to the input parameters.

10. The computing device of claim 9, wherein the processor is further configured to train the estimation blocks, such that each estimation block among the estimation blocks provides an output parameter from among the output parameters in response to the input parameters corresponding to the sample input data.

11. The computing device of claim 9, wherein the processor includes a plurality of processors, and a plurality of essential input parameters is selected in parallel using the plurality of processors in relation to the estimation blocks.

12. The computing device of claim 9, wherein each of the input parameters is classified into an input parameter group from among a plurality of input parameter groups,
each of the output parameters is classified into an output parameter group from among a plurality of output parameter groups respectively corresponding to the input parameter groups, and
each of the estimation blocks is classified into an estimation block group from among a plurality of estimation block groups respectively corresponding to the plurality of output parameter groups.

13. The computing device of claim 12, wherein the processor is further configured to sequentially generate the optimal input data, if no correlation exists between the input parameter groups, else the processor is further configured to recursively generate the optimal input data, if a correlation exists between the input parameter groups.

14. The computing device of claim 1, wherein the processor is further configured to retrain the estimation model in accordance with the essential input data and the sample output data, and generate recommendation input data in accordance with an acquisition function using the estimation model following retraining of the estimation model.

15. The computing device of claim 14, wherein the processor is further configured to determine whether termination conditions have been satisfied, and
if the termination conditions have been satisfied, set recommendation data yielding maximum value for the acquisition function as the optimal input data, else if the termination conditions have not been satisfied, generate recommendation output data from the recommendation input data, and retrain the estimation model using the recommendation data including both the recommendation input data and the recommendation output data.

16. The computing device of claim 15, wherein the processor is further configured to generate recommendation input data following retraining of the estimation model using the recommendation data, and again determine whether termination conditions have been satisfied.

17. A method of generating optimal input data for a design simulator providing output data related to output parameters in response to input data related to input parameters, the method comprising:
generating training data including sample input data and sample output data;
selecting at least one essential input parameter affecting a plurality of output parameters from among the input parameters in accordance with an estimation model trained using the training data; and
generating the optimal input data in accordance with essential input data corresponding to the at least one essential input parameter and the sample output data.

18. The method of claim 17, wherein the at least one essential input parameter has a non-zero input weight, and
the selecting of the at least one essential input parameter includes training the estimation model using the sample input data and the sample output data, on which weigh data is reflected, and select the essential input parameter.

19. The method of claim 18, wherein the generating of the optimal input data includes:
retraining the estimation model using the essential input data and the sample output data;
determining whether termination conditions have been satisfied; and
if the termination conditions have been satisfied, generating recommendation input data yielding maximum value for an acquisition function according to the estimation model following retraining of the estimation model using recommendation data including the recommendation input data and recommendation output data generated from the recommendation input data.

20. Non-transitory storage medium, when executed by at least one processor, storing instructions for the at least one processor to perform a method generating optimal input data for a design simulator providing output data related to output parameters in response to input data related to input parameters,
wherein the method comprises:
generating training data including sample input data and sample output data;
selecting at least one essential input parameter affecting a plurality of output parameters from among a plurality of input parameters in accordance with an estimation model trained using the training data; and
generating the optimal input data using essential input data corresponding to the at least one essential input parameter and the sample output data.
